(19) United States
(12) Patent Application Publication
(10) Pub. No.: US 2025/0000389 A1
(43) Pub. Date: Jan. 2, 2025

(54) AN APPARATUS FOR PRODUCING INFORMATION INDICATIVE OF CARDIAC ABNORMALITY

(71) Applicant: PRECORDIOR OY, TURKU (FI)

(72) Inventors: Juuso BLOMSTER, TURKU (FI); Kamal Jafarian DEHKORDI, TURKU (FI)

(21) Appl. No.: 18/292,745

(22) PCT Filed: Oct. 6, 2023
(86) PCT No.: PCT/FI2023/050576

§ 371 Date: Jan. 26, 2024

(30) Foreign Application Priority Data
Dec. 9, 2022 (FI) 20226089

(51) Int. Cl. A61B 5/11; A61B 5/00

(52) U.S. Cl. CPC A61B 5/1121; A61B 5/7264; A61B 5/7271; A61B 2562/0219

(57) ABSTRACT

An apparatus for producing information indicative of a heart failure with reduced ejection fraction HFrEF includes a signal interface for receiving a signal indicative of cardiac angular rotations and a processing system coupled to the signal interface. The processing system is configured to extract, from the signal, temporal portions which belongs to diastolic phases of a heart. The processing system is configured to set an output signal of the apparatus to express presence of a heart failure with reduced ejection fraction based on a result of a comparison between the indicator quantity and a threshold value.

### FIELD OF THE DISCLOSURE

The disclosure relates generally to producing information indicative of a heart failure with reduced ejection fraction “HFrEF”. More particularly, the disclosure relates to an apparatus for producing information indicative of HFrEF. Furthermore, the disclosure relates to a computer program for producing information indicative of HFrEF.

### BACKGROUND

Abnormalities that may occur in the cardiovascular system, if not diagnosed and appropriately treated and/or remedied, may progressively decrease the ability of the cardiovascular system to maintain a blood flow that meets the needs of a body of an individual especially when the individual encounters physical stress. For example, coronary flow reserve “CFR” is reduced not only because of ischemic heart diseases, but also due to heart failures “HF” which are an increasingly significant global health challenge, imposing major economic liability and health care burden due to high hospitalization, morbidity, and mortality rates. Although HF is defined as a syndrome characterized by symptoms and findings on physical examination, it may be further differentiated based on left ventricular ejection fraction “LVEF” and categorized as HF with reduced ejection fraction “HFrEF”, also known as systolic heart failure, and HF with preserved ejection fraction “HFpEF”. The presenting clinical syndromes in HFpEF and HFrEF are similar but mortality differs, being lower in HFpEF than in HFrEF. In a case of HFrEF, the heart muscle is not able to contract adequately and, therefore, expels less oxygen-rich blood into the body.

Currently, methods such as cardiography based on electromagnetic phenomena related to cardiac activity, echocardiography, and cardiography based on cardiac motion are used in the identification and assessment of various cardiac abnormalities. A well-known example of the cardiography based on electromagnetic phenomena related to cardiac activity is the electrocardiograma, and examples of the cardiography based on cardiac motion are gyrocardiography “GCG” and seismocardiography “SCG”. The echocardiography is typically based on ultrasound and provides images of sections of the heart and can provide comprehensive information about the structure and function of the heart but requires expensive equipment and specialised operating personnel. The ECG provides a rapid electrical assessment of the heart but provides only little information relating to the structure of the heart. The Seismocardiography is a non-invasive accelerometer-based method where precordial vibrations of the heart are measured, while gyrocardiography is a non-invasive gyroscope-based method where cardiac angular rotations are measured. In this document, the term “gyroscope” covers sensors of various kinds for measuring angular rotations.

The above-mentioned heart failure with reduced ejection fraction “HFrEF” can be relatively straightforwardly detected with ultrasound-based echocardiography. There is however still a need for HFrEF detection methods that can be implemented with commonly used consumer products such as for example a smartphone to enable a user to make a first assessment of possible HFrEF at home and send a detection result to a health care system for investigation.

### SUMMARY

The following presents a simplified summary to provide a basic understanding of some aspects of various invention embodiments. The summary is not an extensive overview of the invention. It is neither intended to identify key or critical elements of the invention nor to delineate the scope of the invention. The following summary merely presents some concepts of the invention in a simplified form as a prelude to a more detailed description of exemplifying embodiments of the invention.

In accordance with the invention, there is provided a new apparatus for producing information indicative of a heart failure with reduced ejection fraction “HFrEF”. The apparatus according to the invention comprises a signal interface for receiving a signal indicative of cardiac angular rotations and a processing system coupled to the signal interface. The processing system is configured to:
extract, from the signal, temporal portions which belongs to diastolic phases of a heart,
form an indicator quantity indicative of energy of the temporal portions belonging to the diastolic phases, and
set an output signal of the apparatus to express presence of a heart failure with reduced ejection fraction “HFrEF” based on a result of a comparison between the indicator quantity and a threshold value.

In the light of empirical data, the above-mentioned indicator quantity indicative of the above-mentioned energy related to diastolic phases can be used as an indicator of a heart failure with reduced ejection fraction “HFrEF”.

The above-mentioned threshold value which is compared to the indicator quantity can be determined based on empirical data gathered from a group of patients and healthy persons. The threshold value is not necessary constant, but the threshold value can be changing according to the individual under consideration, according to time, and/or according to some other factors. It is also possible to construct a series of threshold values where each threshold value represents a specific probability of HFrEF.

The apparatus may comprise a sensor system for measuring the signal indicative of cardiac angular rotations. The sensor system may comprise a gyroscope for measuring cardiac angular rotations. It is also possible that the sensor system comprises an accelerometer for measuring cardiac accelerations in different directions and the apparatus comprises a processor for computationally estimating the cardiac angular rotations based on accelerations measured in different directions. Mathematics of orientation determination using a three-axis accelerometer is presented for example by Pedley M.: Tilt Sensing Using a Three-Axis Accelerometer, Freescale Semiconductor Application Note, Document Number: AN3461, Rev. 6, March 2013.

An apparatus according to an exemplifying and non-limiting embodiment can be for example a smartphone or another hand-held device comprising a gyroscope and/or accelerometers. The apparatus can be placed on an individual's chest to measure signals caused by heartbeats.

It is also possible that the signal interface of an apparatus according to an exemplifying and non-limiting embodiment is capable of receiving the signal from an external device comprising an appropriate sensor system, i.e. it is to be noted that the apparatus does not necessarily comprise means for measuring the signal indicative of cardiac angular rotations.

In accordance with the invention, there is also provided a new computer program for producing information indicative of cardiac abnormality based on the above-mentioned signal indicative of cardiac angular rotations. The computer program comprises computer executable instructions for controlling a programmable processing system to:
extract, from the signal, temporal portions which belongs to diastolic phases of a heart,
form an indicator quantity indicative of energy of the temporal portions belonging to the diastolic phases, and
set an output signal to express presence of a heart failure with reduced ejection fraction “HFrEF” based on a result of a comparison between the indicator quantity and a threshold value.

In accordance with the invention, there is also provided a new computer program product. The computer program product comprises a non-volatile computer readable medium, e.g. a compact disc “CD”, encoded with a computer program according to the invention.

Various exemplifying and non-limiting embodiments are described in accompanied dependent claims.

Various exemplifying and non-limiting embodiments both as to constructions and to methods of operation, together with additional objects and advantages thereof, will be best understood from the following description of specific exemplifying embodiments when read in conjunction with the accompanying drawings.

The verbs “to comprise” and “to include” are used in this document as open limitations that neither exclude nor require the existence of also un-recited features.

The features recited in the accompanied dependent claims are mutually freely combinable unless otherwise explicitly stated.

Furthermore, it is to be understood that the use of “a” or “an”, i.e. a singular form, throughout this document does not exclude a plurality.

### BRIEF DESCRIPTION OF FIGURES

Exemplifying and non-limiting embodiments and their advantages are explained in greater detail below with reference to the accompanying drawings, in which:

FIG. 1 shows a schematic illustration of an apparatus according to an exemplifying and non-limiting embodiment for producing information indicative of a heart failure with reduced ejection fraction “HFrEF”, and

FIG. 2 shows true positive rate vs. false positive rate-curves which are related to detecting a heart failure with reduced ejection fraction “HFrEF” based on signals indicative of cardiac angular rotations measured with a gyroscope and on signals indicative of cardiac accelerations measured with an accelerometer.

### DESCRIPTION OF EXEMPLIFYING AND NON-LIMITING EMBODIMENTS

The specific examples provided in the description below should not be construed as limiting the scope and/or the applicability of the appended claims. Lists and groups of examples provided in the description are not exhaustive unless otherwise explicitly stated.

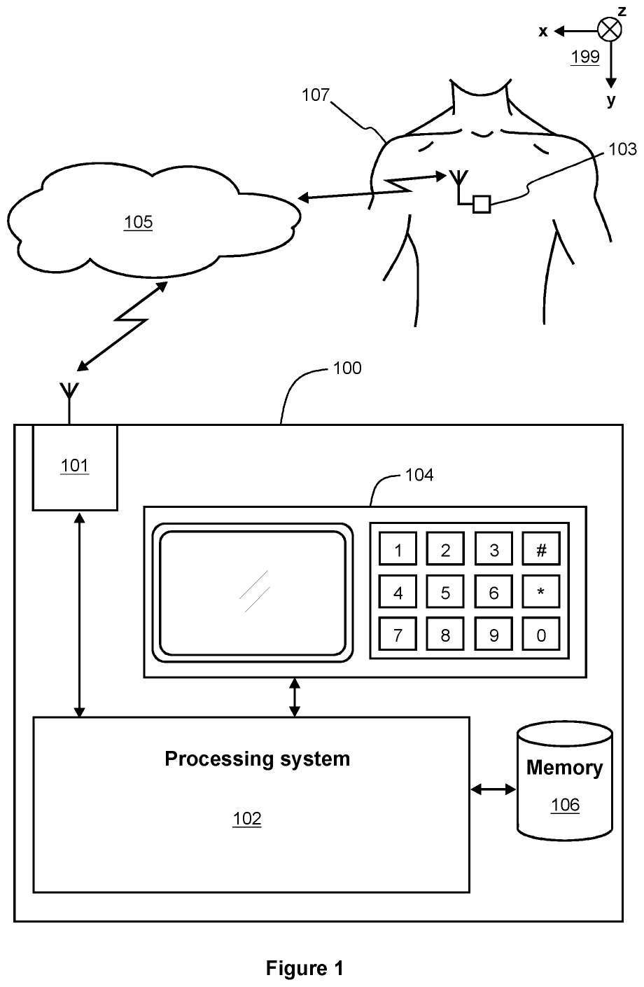

FIG. 1 shows a schematic illustration of an apparatus 100 according to an exemplifying and non-limiting embodiment for producing information indicative of a heart failure with reduced ejection fraction “HFrEF”. The apparatus comprises a signal interface 101 for receiving a signal indicative of cardiac angular rotations and a processing system 102 coupled to the signal interface 101. The processing system 102 is configured to:
extract, from the signal, temporal portions which belongs to diastolic phases of a heart,
form an indicator quantity indicative of energy of the temporal portions belonging to the diastolic phases, and
set an output signal of the apparatus to express presence of HFrEF based on a result of a comparison between the indicator quantity and a threshold value.

The above-mentioned signal is produced with a sensor system 103 that is responsive to cardiac angular rotations. In the exemplifying situation shown in FIG. 1, the sensor system 103 is placed on the chest of an individual 107. The sensor system 103 may comprise for example a gyroscope and/or an inertial measurement unit “IMU” comprising both an accelerometer and a gyroscope. The sensor system 103 can be for example a microelectromechanical system “MEMS”. The gyroscope can be for example a Coriolis vibratory gyroscope “CVG” that uses a vibrating structure to determine a rate of rotation. The temporal duration of the signal measured with the sensor system can be, for example but not necessarily, from tens of seconds to hours. The output signal of the apparatus can be for example a message shown on a display screen of a user-interface 104. The temporal portions which belong to the diastolic phases can be recognized and extracted from the signal with suitable known methods that can be based on e.g. known waveform complexes which are related to the systolic phase and to the diastolic phase, respectively.

In the exemplifying case illustrated in FIG. 1, the sensor system 103 is connected to the signal interface 101 via one or more data transfer links each of which can be for example a radio link or a corded link. The data transfer from the sensor system 103 to the signal interface 101 may take place either directly or via a data transfer network 105 such as e.g. a telecommunications network. In the exemplifying case illustrated in FIG. 1, the sensor system 103 is connected to a radio transmitter. It is also possible that the apparatus comprising the processing device 102 is integrated with the sensor system. In this exemplifying case, the signal interface is a simple wiring from the sensor system to the processing device. An apparatus comprising an integrated sensor system can be for example a smartphone or another hand-held device which can be placed on the chest of an individual during a measurement phase.

An apparatus according to an exemplifying and non-limiting embodiment is configured to record the signal indicative of cardiac angular rotations. The recorded signal can be measured within a time window having a fixed temporal start-point and a fixed temporal endpoint or within a sliding time window having a fixed temporal length and moving along with elapsing time. The apparatus may comprise an internal memory 106 for recording the signal and/or the apparatus may comprise a data port for connecting to an external memory.

There are numerous ways to form the indicator quantity indicative of the energy related to the diastolic phases of heart-beat periods. In an apparatus according to an exemplifying and non-limiting embodiment, the processing system 102 is configured to compute the indicator quantity according to the following formula:

$${\sum }_{i = 1}^{N} ( {x}_{i}^{2} + {y}_{i}^{2} + {z}_{i}^{2} ) , \qquad ( 1 )$$

where i is an index increasing with time, N is the number of samples taken from the signal during the diastolic phases of heart-beat periods, xᵢ is an i^{th} sample of cardiac rotation with respect to the x-direction of a cartesian coordinate system 199, yᵢ is an i^{th} sample of cardiac rotation with respect to the y-direction of the cartesian coordinate system 199, and zᵢ is an i^{th} sample of cardiac rotation with respect to the z-direction of the cartesian coordinate system 199. In this exemplifying case, the signal indicative of cardiac angular rotations can be received from a three-axis gyroscope and the signal has components indicative of rotations with respect to the x-, y-, and z-directions of the cartesian coordinate system 199.

In an apparatus according to an exemplifying and non-limiting embodiment, the processing system 102 is configured to maintain a series of threshold values where each threshold value represents a specific probability of HFrEF. The processing system 102 is configured to set the output signal of the apparatus to express the probability of HFrEF based on results of comparisons between the indicator quantity and the threshold values.

A heart failure “HR”, both with reduced ejection fraction “HFrEF” and with preserved ejection fraction “HFpEF”, is associated with the third heart sound S₃ that is an extra heart sound that occurs soon after the normal two heart sounds S₁ and S₂ related to aorta opening and aorta closure. The third heart sound S₃ occurs at the beginning of the middle third of diastole, approximately 0.12 to 0.18 seconds after S₂. The third heart sound S₃ can be thought to be caused by the oscillation of blood back and forth between the walls of the ventricles initiated by inflow of blood from the atria. The reason for that the third heart sound S₃ does not occur until the middle third of diastole is probably that, during the early part of diastole, the ventricles are not filled sufficiently to create enough tension for reverberation. It may also be a result of tensing of the chordae tendineae during rapid filling and expansion of the ventricle.

In an apparatus according to an exemplifying and non-limiting embodiment, the processing system 102 is configured to extract, from the signal, the temporal portions so that each of the extracted temporal portions represents a middle third of a corresponding diastolic phase.

The processing system 102 can be implemented for example with one or more processor circuits, each of which can be a programmable processor circuit provided with appropriate software, a dedicated hardware processor such as, for example, an application specific integrated circuit “ASIC”, or a configurable hardware processor such as, for example, a field programmable gate array “FPGA”. The memory 106 can be implemented for example with one or more memory circuits, each of which can be e.g. a random-access memory “RAM” device.

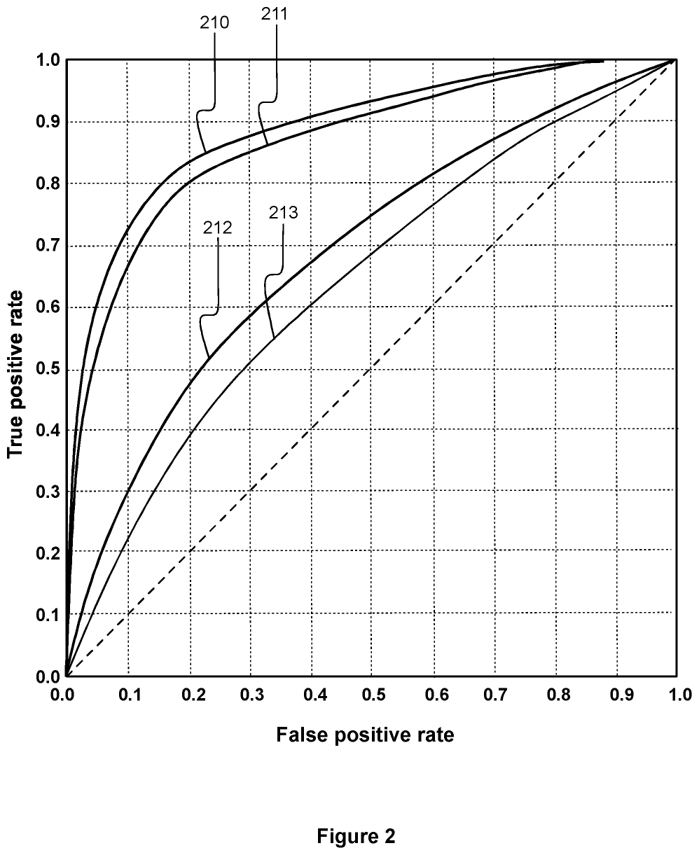

FIG. 2 shows curves 210 and 211 which express a true positive rate vs. a false positive rate. These curves 210 and 211 are related to detecting a heart failure with reduced ejection fraction “HFrEF” based on signals which are indicative of cardiac angular rotations, and which have been measured with a gyroscope. The curve 210 relates to a situation in which there is atrial fibrillation, and the curve 210 relates to a situation in which there is no atrial fibrillation.

In the example cases related to the curves 210 and 211 in FIG. 2, the gyroscope is a three-axis gyroscope, and the signal indicative of cardiac angular rotations has components indicative of rotations with respect to the x-, y-, and z-directions of a cartesian coordinate system. The indicator quantity is computed according to the above-presented formula 1, and a detection result is set to express presence of a heart failure with reduced ejection fraction “HFrEF” in response to a situation in which the indicator quantity exceeds a threshold value. The true positive rate, also called sensitivity, is the probability that an actual positive, i.e. real presence of HFrEF, will be detected as positive. The false positive rate is the probability that a false alarm will be raised i.e. a positive result is detected when the true value is negative i.e. there is no HFrEF. When the above-mentioned threshold value is decreased, the true positive rate i.e. the probability that real presence of HFrEF will be detected as positive is increased but the false positive rate i.e. the probability that a false alarm will be raised is increased, too.

Furthermore, FIG. 2 shows curves 212 and 213 which express a true positive rate vs. a false positive rate so that the curve 212 relates to a situation where there is atrial fibrillation, and the curve 213 relates to a situation where there is no atrial fibrillation. These curves 212 and 213 are related to detecting a heart failure with reduced ejection fraction “HFrEF” based on signals which are indicative of cardiac accelerations, and which have been measured with an accelerometer. In this exemplifying case, the accelerometer is a three-axis accelerometer, and the signal indicative of cardiac accelerations has components indicative of accelerations in the x-, y-, and z-directions of a cartesian coordinate system. The indicator quantity is computed in a way corresponding to the above-presented formula 1, and a detection result is set to express presence of a heart failure with reduced ejection fraction “HFrEF” in response to a situation in which the indicator quantity exceeds a threshold value. When this threshold value is decreased, the true positive rate i.e. the probability that real presence of HFrEF will be detected as positive is increased but the false positive rate i.e. the probability that a false alarm will be raised is increased, too.

As shown by FIG. 2, the detection of HFrEF based on measurements of cardiac angular rotations provide significantly better true positive rates with smaller false positive rates than the detection of HFrEF based on measurements of cardiac accelerations. The measurements of cardiac angular rotations provide better detection results both in the situation where there is atrial fibrillation as well as in the situation where there is no atrial fibrillation.

A computer program according to an exemplifying and non-limiting embodiment comprises software modules for producing information indicative of a heart failure with reduced ejection fraction “HFrEF” based on a signal indicative of cardiac angular rotations. The software modules comprise computer executable instructions for controlling a programmable processing system to:
extract, from the signal, temporal portions which belongs to diastolic phases of a heart,
form an indicator quantity indicative of energy of the temporal portions belonging to the diastolic phases, and
set an output signal to express presence of HFrEF based on a result of a comparison between the indicator quantity and a threshold value.

In a computer program according to an exemplifying and non-limiting embodiment, the software modules comprise computer executable instructions for controlling the programmable processing system to compute the indicator quantity according to the above-presented formula 1.

In a computer program according to an exemplifying and non-limiting embodiment, the software modules comprise computer executable instructions for controlling the programmable processing system to extract, from the signal, the temporal portions so that each of the extracted temporal portions represents a middle third of a corresponding diastolic phase.

The software modules can be e.g. subroutines or functions implemented with a suitable programming language and with a compiler suitable for the programming language and for the programmable processing system under consideration. It is worth noting that also a source code corresponding to a suitable programming language represents the computer executable software modules because the source code contains the information needed for controlling the programmable processing system to carry out the above-presented actions and compiling changes only the format of the information. Furthermore, it is also possible that the programmable processing system is provided with an interpreter so that a source code implemented with a suitable programming language does not need to be compiled prior to running.

A computer program product according to an exemplifying and non-limiting embodiment comprises a computer readable medium, e.g. a compact disc “CD”, encoded with a computer program according to an embodiment of invention.

A signal according to an exemplifying and non-limiting embodiment is encoded to carry information defining a computer program according to an embodiment of invention.

The specific examples provided in the description given above should not be construed as limiting the scope and/or the applicability of the appended claims. Lists and groups of examples provided in the description given above are not exhaustive unless otherwise explicitly stated.

1. An apparatus comprising:
a signal interface for receiving a signal indicative of cardiac angular rotations, and
a processing system coupled to the signal interface,
wherein the processing system is configured to:
extract, from the signal, temporal portions which belongs to diastolic phases of a heart,
form an indicator quantity indicative of energy of the temporal portions belonging to the diastolic phases, and
set an output signal of the apparatus to express presence of a heart failure with reduced ejection fraction based on a result of a comparison between the indicator quantity and a threshold value.

2. The apparatus according to claim 1, wherein the apparatus comprises a sensor system for producing the signal indicative of the cardiac angular rotations.

3. The apparatus according to claim 2, wherein the sensor system comprises a gyroscope for measuring the cardiac angular rotations.

4. The apparatus according to claim 3, wherein the gyroscope is a Coriolis vibratory gyroscope comprising a vibrating structure to determine a rate of rotation.

5. The apparatus according to claim 1, wherein the processing system is configured to compute the indicator quantity according to the formula:
$${\sum }_{i = 1}^{N} ( {x}_{i}^{2} + {y}_{i}^{2} + {z}_{i}^{2} ) ,$$
where i is an index increasing with time, N is a number of samples of the temporal portions belonging to the diastolic phases, xᵢ is an i^{th} sample of cardiac rotation with respect to an x-direction of a cartesian coordinate system, yᵢ is an i^{th} sample of cardiac rotation with respect to a y-direction of the cartesian coordinate system, and zᵢ is an i^{th} sample of cardiac rotation with respect to a z-direction of the cartesian coordinate system.

6. The apparatus according to claim 1, wherein the processing system is configured to extract, from the signal, the temporal portions so that each of the extracted temporal portions represents a middle third of the corresponding diastolic phase.

7. A non-transitory computer readable medium encoded with a computer program for controlling a programmable processing system to process a signal indicative of cardiac angular rotations, the computer program comprising computer executable instructions for controlling the programmable processing system to:
extract, from the signal, temporal portions which belongs to diastolic phases of a heart,
form an indicator quantity indicative of energy of the temporal portions belonging to the diastolic phases, and
set an output signal to express presence of a heart failure with reduced ejection fraction based on a result of a comparison between the indicator quantity and a threshold value.

8. The non-transitory computer readable medium according to claim 7, wherein the computer program comprises computer executable instructions for controlling the programmable processing system to compute the indicator quantity according to the formula:
$${\sum }_{i = 1}^{N} ( {x}_{i}^{2} + {y}_{i}^{2} + {z}_{i}^{2} ) ,$$
where i is an index increasing with time, N is a number of samples of the temporal portions belonging to the diastolic phases, xᵢ is an i^{th} sample of cardiac rotation with respect to an x-direction of a cartesian coordinate system (199), yᵢ is an i^{th} sample of cardiac rotation with respect to a y-direction of the cartesian coordinate system, and zᵢ is an i^{th} sample of cardiac rotation with respect to a z-direction of the cartesian coordinate system.

9. The non-transitory computer readable medium according to claim 7, wherein the computer program comprises computer executable instructions for controlling the programmable processing system to extract, from the signal, the temporal portions so that each of the extracted temporal portions represents a middle third of the corresponding diastolic phase.

10. (canceled)

11. The apparatus according to claim 2, wherein the processing system is configured to compute the indicator quantity according to the formula:
$${\sum }_{i = 1}^{N} ( {x}_{i}^{2} + {y}_{i}^{2} + {z}_{i}^{2} ) ,$$
where i is an index increasing with time, N is a number of samples of the temporal portions belonging to the diastolic phases, xᵢ is an i^{th} sample of cardiac rotation with respect to an x-direction of a cartesian coordinate system, yᵢ is an i^{th} sample of cardiac rotation with respect to a y-direction of the cartesian coordinate system, and zᵢ is an i^{th} sample of cardiac rotation with respect to a z-direction of the cartesian coordinate system.

12. The apparatus according to claim 3, wherein the processing system is configured to compute the indicator quantity according to the formula:
$${\sum }_{i = 1}^{N} ( {x}_{i}^{2} + {y}_{i}^{2} + {z}_{i}^{2} ) ,$$
where i is an index increasing with time, N is a number of samples of the temporal portions belonging to the diastolic phases, xᵢ is an i^{th} sample of cardiac rotation with respect to an x-direction of a cartesian coordinate system, yᵢ is an i^{th} sample of cardiac rotation with respect to a y-direction of the cartesian coordinate system, and zᵢ is an i^{th} sample of cardiac rotation with respect to a z-direction of the cartesian coordinate system.

13. The apparatus according to claim 4, wherein the processing system is configured to compute the indicator quantity according to the formula:
$${\sum }_{i = 1}^{N} ( {x}_{i}^{2} + {y}_{i}^{2} + {z}_{i}^{2} ) ,$$
where i is an index increasing with time, N is a number of samples of the temporal portions belonging to the diastolic phases, xᵢ is an i^{th} sample of cardiac rotation with respect to an x-direction of a cartesian coordinate system, yᵢ is an i^{th} sample of cardiac rotation with respect to a y-direction of the cartesian coordinate system, and zᵢ is an i^{th} sample of cardiac rotation with respect to a z-direction of the cartesian coordinate system.

14. The apparatus according to claim 2, wherein the processing system is configured to extract, from the signal, the temporal portions so that each of the extracted temporal portions represents a middle third of the corresponding diastolic phase.

15. The apparatus according to claim 3, wherein the processing system is configured to extract, from the signal, the temporal portions so that each of the extracted temporal portions represents a middle third of the corresponding diastolic phase.

16. The apparatus according to claim 4, wherein the processing system is configured to extract, from the signal, the temporal portions so that each of the extracted temporal portions represents a middle third of the corresponding diastolic phase.

17. The apparatus according to claim 5, wherein the processing system is configured to extract, from the signal, the temporal portions so that each of the extracted temporal portions represents a middle third of the corresponding diastolic phase.

18. The non-transitory computer readable medium according to claim 8, wherein the computer program comprises computer executable instructions for controlling the programmable processing system to extract, from the signal, the temporal portions so that each of the extracted temporal portions represents a middle third of the corresponding diastolic phase.
